(19) United States
(12) Patent Application Publication
(10) Pub. No.: US 2016/0324679 A1
(43) Pub. Date: Nov. 10, 2016

(54) Hands-Free Apparatus for Collecting Nasal Discharge

(71) Applicant: Arshad Majid Khan, Wilmette, IL (US)

(72) Inventor: Arshad Majid Khan, Wilmette, IL (US)

(21) Appl. No.: 15/145,783

(22) Filed: May 3, 2016

Related U.S. Application Data
(60) Provisional application No. 62/179,309, filed on May 4, 2015.

(51) Int. Cl. A61F 5/44; A61F 5/443; A61F 13/20

(52) U.S. Cl. CPC A61F 5/4401; A61F 13/2005; A61F 5/443

(57) ABSTRACT

A hands-free apparatus for collecting nasal discharge from a runny nose is provided. The apparatus includes a first and second absorbent masses supported at each end of a flexible horse-shoe shaped member. The adjustable orientation of the first and second absorbent masses at the ends of the flexible horse-shoe shaped member allows the first absorbent mass to be inserted into the left nostril of an individual's nose and the second absorbent mass to be inserted into the right nostril of the individual's nose, and to be retained therein, in order to collect fluid emanating from the nose.

### CROSS-REFERENCE TO RELATED APPLICATION

This application claims the benefit of U.S. Provisional Application No. 62/179,309, filed May 4, 2015.

### BACKGROUND OF THE INVENTION

When one has a cold, allergies or other upper respiratory condition, or when one is simply outside in the cold, one's nose tends run. The most common method for dealing with a runny nose is to wipe away the flowing discharge using a tissue or handkerchief. Blowing one's nose into a tissue or handkerchief may also provide temporary respite from the constant drip of a runny nose by collecting a portion of the nasal discharge that has built up in one's nasal passages. These solutions, while providing temporary relief, suffer a number of drawbacks. Blowing one's nose, for example, requires the use of one or both of the individual's hands, interfering with activities the individual may be otherwise engaged in with his or her hands. One must also dispose of the used tissue or deal with a soiled handkerchief. Furthermore, the relief provided by wiping or blowing one's nose is purely temporary. After a short reprieve the individual's nose will invariably begin to run again so long as the underlying causes of the runny nose (sickness, allergies, etc.) persist.

### SUMMARY OF THE INVENTION

A hands-free apparatus for collecting nasal discharge from a runny nose is provided. The hands-free apparatus for collecting nasal discharge includes a first absorbent mass supported at a first end of a flexible arcuate member and a second absorbent mass supported at the opposite end of the flexible arcuate member. The flexible arcuate member supports the first and second absorbent masses an adjustable distance apart. The distance between the two absorbent masses generally corresponds to the separation distance between the left and right nostrils of a human nose. The distance separating the two absorbent masses may be adjusted by pinching the two absorbent masses together, flexing the arcuate member to move the two absorbent masses closer together. The adjustable orientation of the first and second absorbent masses at the ends of the flexible arcuate member allows the first absorbent mass to be inserted into the left nostril of an individual's nose and the second absorbent mass to be inserted into the right nostril of the individual's nose and to be retained therein to collect nasal discharge emanating from the nose.

### BRIEF DESCRIPTION OF THE DRAWINGS

FIG. 1 is a plan view of a hands-free apparatus for capturing nasal discharge according to an embodiment of the invention;

FIG. 2 is an illustration of a hands-free apparatus for capturing nasal discharge worn by a user;

FIG. 3 is a detailed view of one end of a hands-free apparatus for capturing nasal discharge according to an embodiment of the invention;

FIG. 4 is a exploded view of one end of a hands-free apparatus for capturing nasal discharge according to another embodiment of the invention;

FIG. 5 is a partial cross sectional view of one end of a hands-free apparatus for capturing nasal discharge according to the embodiment of FIG. 4; and

FIG. 6 is a detailed view of one end of a hands-free apparatus for capturing nasal discharge according to yet another embodiment of the invention.

### DETAILED DESCRIPTION OF THE PREFERRED EMBODIMENTS

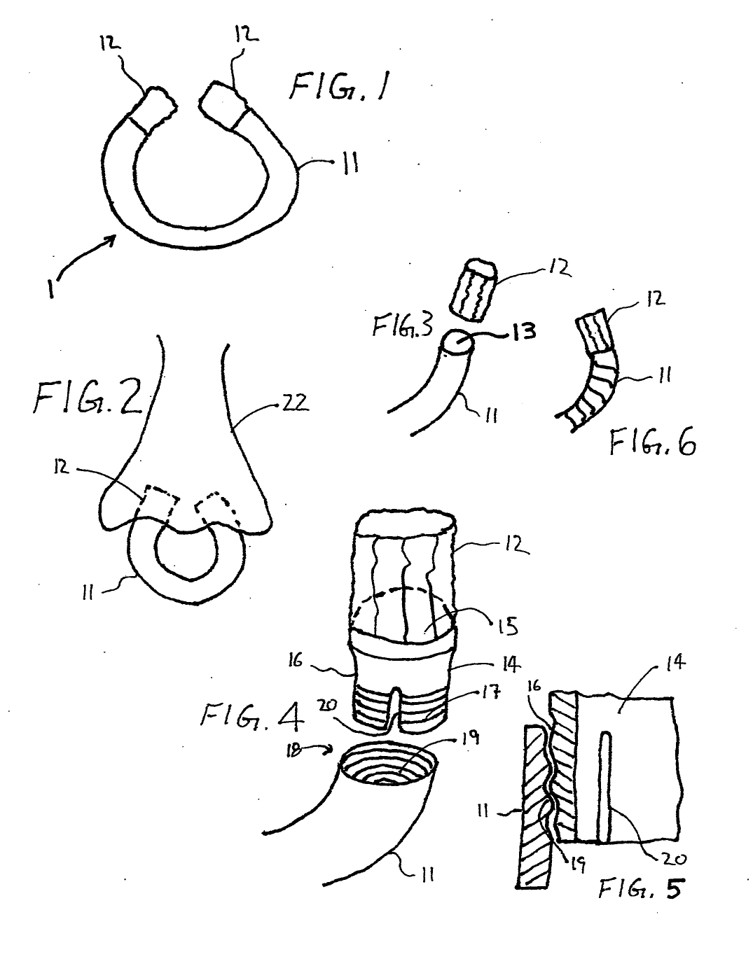

FIG. 1 shows an embodiment of a hands-free apparatus for capturing nasal discharge 1 according to a first embodiment of the invention. The apparatus 1 comprises a generally horse-shoe shaped arcuate member 11 disposed between a pair of absorbent masses 12. The arcuate member 11 may be formed from any suitable material such as cardboard, rolled paper, plastic or any other semi-rigid yet flexible material. The absorbent masses 12 may comprise a mass of natural or synthetic materials such as cotton fibers, tissue paper, synthetic sponge material, or the like.

FIG. 2 shows a hands-free apparatus for capturing nasal discharge 1 in use. The absorbent masses 22 are inserted into each nostril of the user's nose 22. When inserted into the nose 20, the absorbent masses 12 frictionally engage inner surfaces of each nostril to hold the apparatus 1 in place. The arcuate member 11 is sufficiently flexible to allow the user to adjust the spacing between the absorbent masses 12 to accommodate the unique geometry of the individual user's nose. The absorbent masses 12 may be sufficiently porous and sized appropriately to allow air to flow through and around the absorbent masses 12 so that the user may breathe freely while wearing the apparatus 1.

Alternatively, the absorbent masses 12 may be formed from a heavier, more absorbent material for situations in which the user's congestion is worse. In such cases the user's airways may already be blocked, and the free flow of air through the apparatus 1 is not required. In another embodiment passages or grooves may be formed within the absorbent masses 12 to allow for the free flow of air through and around the absorbent masses to allow the user to breathe while wearing the apparatus 1.

The arcuate member 11 may be a solid shaft, a hollow tube, or a partially solid shaft with hollow tubular ends. FIG. 3 shows an exploded detailed view of one end of an apparatus 1 according to an embodiment of the invention. In this embodiment the arcuate member 11 comprises a solid shaft. The end of the shaft provides a solid surface 13 onto which the absorbent mass 12 may be attached using an adhesive.

FIG. 4 shows an exploded detailed view of one end of an apparatus for collecting nasal discharge 1 according to an alternative embodiment of the invention. In the embodiment of FIG. 4 the horse-shoe shaped arcuate member 11 is a hollow tube made of plastic or some other flexible material. The absorbent mass 12 is attached to a mounting plug 14 that may be inserted into the hollow end 18 of arcuate member 11. The mounting plug 14 includes a mounting surface 15 and a tapered lower portion 16 adapted to be inserted into the hollow end 18 of the arcuate member 11. Ridges or threads 17 may be formed on the outer surface of the base portion 16 to frictionally engage inner surfaces of the hollow end 18 of arcuate member 11 or to rotatably engage matching threads (not shown) formed on the inner surfaces of the hollow end 18 arcuate member 11 to hold the mounting plug 16 and the attached absorbent mass 12 in place at the end of the arcuate member 11.

One or more compression slots 20 may be formed in the lower portion 16 of the mounting plug 14. The compression slots may provide a degree of flexibility to the lower portion 16 of the mounting plug 14 for easing the insertion of the lower portion 16 of the mounting plug 14 into the hollow end 18 of the arcuate member 11. While the mounting plug 14 is being inserted into the hollow end 18 of the arcuate member 11, the one or more compression slots 20 allow the portions of the lower base portion 16 mounting plug between the compressions slots 20 to flex inward so that the ridges and grooves 17 formed on the lower portion 16 of the mounting plug 14 may slide past corresponding ridges and grooves 19 formed on the inner surfaces of the hollow end 18 of the arcuate member 11. Once the lower portion 16 of the mounting plug 14 has been fully inserted into the hollow end 18 of the arcuate member 11, the compressible slots 20 decompress, causing the lower portion 16 of the mounting plug 14 to expand outward to its natural state. The ridges and grooves 17 formed on the lower portion 16 of the mounting plug 14 mate with the corresponding ridges and grooves 19 formed on the inner surface of the hollow end 18 of the arcuate member 11 to hold the mounting plug in place, as shown in the detailed cross sectional view of FIG. 5.

In an alternative embodiment (not shown) the mounting plug 16 may itself be hollow, with the material comprising the absorbent mass pulled therethrough and extending out either end. The advantage of this embodiment being that the absorbent material comprising the absorbent mass 12 may act as a wick to draw fluid down out of the user's nose and into the hollow portion of the arcuate member 11.

FIG. 6 is a detail view of one end of another embodiment of a hands-free apparatus for collecting nasal discharge 1. In this embodiment the arcuate member 11 and the absorbent masses 12 are formed from a single piece of absorbent material. In this embodiment the arcuate member 11 is given rigidity by tightly winding the absorbent material in a spiral manner as as shown, while allowing the absorbent material at the ends to remain loose and free. This embodiment has the significant advantage of ease of manufacture, but may not be as durable or as long lasting as other embodiments.

The foregoing merely illustrates the principles of the invention. Various modifications and alterations to the described embodiments will be apparent to those skilled in the art in view of the teachings herein. It will thus be appreciated that those skilled in the art will be able to devise numerous techniques which, although not explicitly described herein, embody the principles of the invention and are thus within scope of the invention, as defined by the claims.

I claim:

1. A hands-free apparatus for collecting nasal discharge, the apparatus comprising:
a first absorbent mass and a second absorbent mass;
a flexible arcuate member disposed between the first and second absorbent masses, the first absorbent mass attached to a first end of the flexible arcuate member and the second absorbent mass attached to a second end of the flexible arcuate member such that the flexible arcuate member supports the first and second absorbent masses an adjustable distance apart, the adjustable distance generally corresponding to a typical separation distance between left and right nostrils of a human nose such that the first absorbent mass may be inserted into the left nostril of a nose while the second absorbent mass may be inserted into the right nostril the nose to collect fluid discharged therefrom.

2. The apparatus of claim 1 wherein the flexible arcuate member is a substantially horse-shoe shaped.

3. The apparatus of claim 1 where the flexible arcuate member comprises a substantially solid shaft and the first and second absorbent masses are attached to first and second ends of the shaft by an adhesive.

4. The apparatus of claim 1 wherein the first and second absorbent masses and the flexible arcuate member are formed from a single piece of absorbent material.

5. The apparatus of claim 4 wherein the a central portion of the single piece of absorbent material is tightly wound into a semi-rigid shaft.

6. The apparatus of claim 5 wherein a central portion of the single piece of absorbent material is compressed into a semi-rigid shaft.

7. The apparatus of claim 1 wherein the flexible arcuate member includes hollow tubular ends, the apparatus further comprising a pair of mounting plugs for attaching the first and second absorbent masses to the arcuate member.

8. The apparatus of claim 7 wherein each mounting plug includes an upper portion for receiving one of the first and second absorbent masses and a lower portion adapted to be attached to one of the hollow ends of the flexible arcuate member.

9. The apparatus of claim 8 wherein mating ridges and grooves are formed within the hollow ends of the flexible arcuate member and on the lower portions of the mounting plugs such that the ridges and grooves formed on the lower portion of the mating plugs may be seated within the ridges and grooves formed within the hollow ends of the flexible arcuate member to retain the mounting plugs and the absorbent masses attached thereto to the ends of the flexible arcuate member.

10. The apparatus of claim 9 wherein one or more compression slots are formed in the lower portion of the mounting plugs.
